(19) United States
(12) Patent Application Publication
(10) Pub. No.: US 2026/0345116 A1
(43) Pub. Date: Nov. 5, 2026

(54) DEVICE FOR HOLDING A MEDICAL CONTROL UNIT AND FACILITY COMPRISING SUCH A DEVICE

(71) Applicant: KOELIS, Meylan (FR)

(72) Inventor: Benjamin DASSONVILLE, Plateau Des Petites Roches (FR)

(73) Assignee: KOELIS, Meylan (FR)

(21) Appl. No.: 19/474,332

(22) PCT Filed: Apr. 10, 2024
(86) PCT No.: PCT/EP2024/059748

§ 371 Date: Oct. 10, 2025

(30) Foreign Application Priority Data
Apr. 13, 2023 (FR) FR2303666

(51) Int. Cl. A61B 8/00; A61B 8/08

(52) U.S. Cl. CPC A61B 8/4209; A61B 8/085

(57) ABSTRACT

A device for holding an examination apparatus. The device includes at least a cradle, a shell mounted on the cradle so as to be movable about an axis of rotation, and a locking member for temporarily securing the cradle to the shell. The locking member includes at least a portion for connecting to the shell, a lock including at least one elastically deformable element, and a handling portion connected to the connection portion and to the lock so as to be able to move the elastically deformable element between an unlocked position. The element is offset from the cradle, and a locked position, in which the element presses against the cradle.

The invention relates to a device for holding a medical examination apparatus.

The invention also relates to an installation comprising such a device.

### BACKGROUND OF THE INVENTION

As a result both of progress in the technologies used and also of good monitoring of the population at risk, cancers of the prostate are being detected earlier and earlier. Consequently, the tumours that are found are getting smaller and smaller.

Conventional treatments of total or partial ablation of the prostate are thus nowadays no longer appropriate, in particular in the light of the severe post-operative consequences to which they give rise (incontinence, impotence, etc.).

Thus, operators are turning increasingly to targeted treatments that involve inserting an instrument into the body of the patient in order to bring the instrument close to a particular region of the prostate for treatment. By way of example, the treatment may be brachytherapy with insertion of a catheter (for positioning radioactive seeds), or it may be other targeted therapies such as cryotherapy or laser therapy.

For the operations to proceed as smoothly as possible, images are acquired continuously by an ultrasound probe that is also inserted into the patient.

Since operators are focused on handling the instrument, the ultrasound probe is carried by a handling arm.

For this purpose, a holding device is used. A device of this kind usually comprises a cradle and a case bearing the ultrasound probe, the case being movably mounted on the cradle.

The holding device further comprises a first wing screw so that the shell can be locked in position in the cradle, and a second wing screw that interacts with a second nut so that the ultrasound probe can be secured to the shell. It is therefore understood that handling the holding device is relatively onerous for operators, which can cause problems, in particular during an operation.

### OBJECT OF THE INVENTION

An object of the invention is to propose a more ergonomic device for holding a medical examination apparatus.

Another object of the invention is to propose an installation comprising such a device.

### SUMMARY OF THE INVENTION

To achieve this object, what is proposed according to the invention is a device for holding an examination apparatus, the device comprising at least a cradle, a shell mounted on the cradle so as to be movable about an axis of rotation, and a locking member for temporarily securing the cradle to the shell.

According to the invention, the locking member comprises at least:
a portion for connecting to the shell,
a lock comprising at least one elastically deformable element,
a handling portion connected to the connection portion and to the lock so as to be able to move the elastically deformable element between an unlocked position, in which said element is offset from the cradle, and a locked position, in which the element presses against the cradle.

The invention thus makes it possible to temporarily secure the cradle to the shell.

This temporary securing is advantageously carried out in a simple and rapid manner. In particular, it does not require any screwing/unscrewing operation.

Optionally, the elastically deformable element is a washer. Optionally, the elastically deformable element is mounted on a hinge pin on which the handling portion acts in order to move the elastically deformable element between its two positions.

Optionally, the handling portion is pivotably mounted on the connection portion.

Optionally, the handling portion comprises a handle extended by a fork joint secured to the handling portion. Optionally, the connection portion comprises a hollow casing and a shaft mounted in the hollow casing.

Optionally, the shaft is mounted in the casing so as to be slidable between a first position and a second position.

Optionally, the shaft is rigidly connected to a hinge pin on which the elastically deformable element is mounted.

Optionally, the casing is provided with an aperture through which the hinge pin rigidly connected to the shaft extends. Optionally, the device comprises a spring connected both to the column and to the shaft.

Optionally, the shell is provided with an aperture through which the column and the shaft extend.

Optionally, the aperture and the column are provided with their temporary securing means.

Optionally, the temporary securing means comprise a peg and at least one recess in which the peg is able to fit temporarily.

Optionally, the device comprises a second locking member for securing the examination apparatus to the shell during operation, the second locking member comprising at least a latch and a lock, the lock comprising at least:
an attachment to the shell,
an element for interacting with the latch, the interacting element being mounted in the device so as to be movable between an active position, in which said element is locked by the latch, and an inactive position in which said element is not locked by the latch,
an arm which is connected at a first end to the attachment and connected at a second end to the interacting element, the arm being intended to hold the examination apparatus against the shell.

The invention also relates to an installation comprising a device as described above.

Other features and advantages of the invention become clear on reading the following description of particular and non-limiting embodiments of the invention.

### BRIEF DESCRIPTION OF THE DRAWINGS

The invention will be better understood in the light of the following description, which is given with reference to the accompanying drawings, in which:

FIG. 1 is a partial schematic perspective view of an installation according to a first embodiment of the invention, the interacting element being in its active position,

FIG. 2 is a partial schematic perspective view of the installation shown in FIG. 1, the interacting element being in its inactive position,

FIG. 3 is a partial sectional view of the installation shown in FIG. 1,

FIG. 4 is a partial schematic perspective view of the installation shown in FIG. 1,

FIG. 5 is a partial schematic sectional view of an installation according to a second embodiment of the invention, the interacting element being in its active position,

FIG. 6 is a partial schematic perspective view of an installation according to a third embodiment of the invention, the interacting element being in its inactive position,

FIG. 7 is a partial schematic perspective side view of the installation according to the first embodiment shown in FIG. 1,

FIG. 8 is a schematic plan view of a shell of the installation shown in FIG. 7, a connection portion of the installation being in its securing position,

FIG. 9 is a similar view to FIG. 8, the connection portion being in its separating position,

FIG. 10 is a similar view to FIG. 8, the connection portion not being shown,

FIG. 11 is another partial schematic sectional view of the installation shown in FIG. 7.

### DETAILED DESCRIPTION OF THE INVENTION

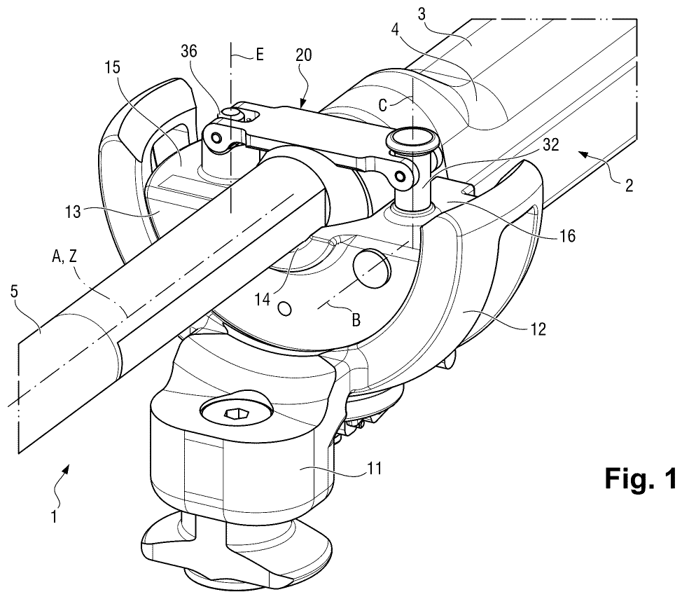

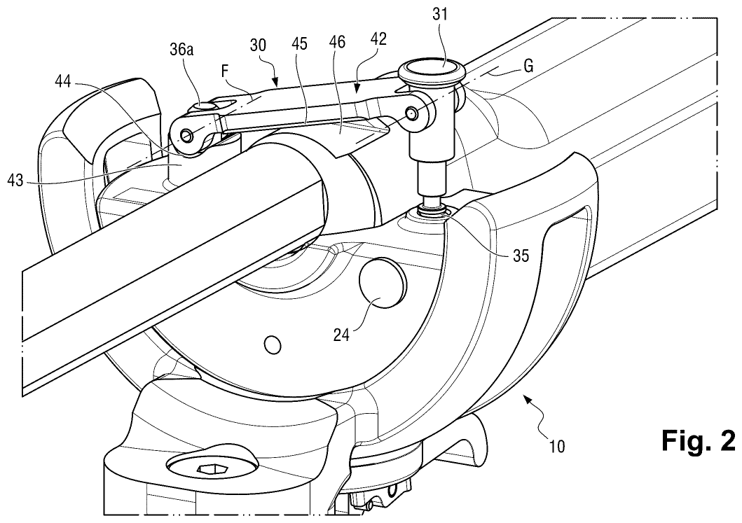

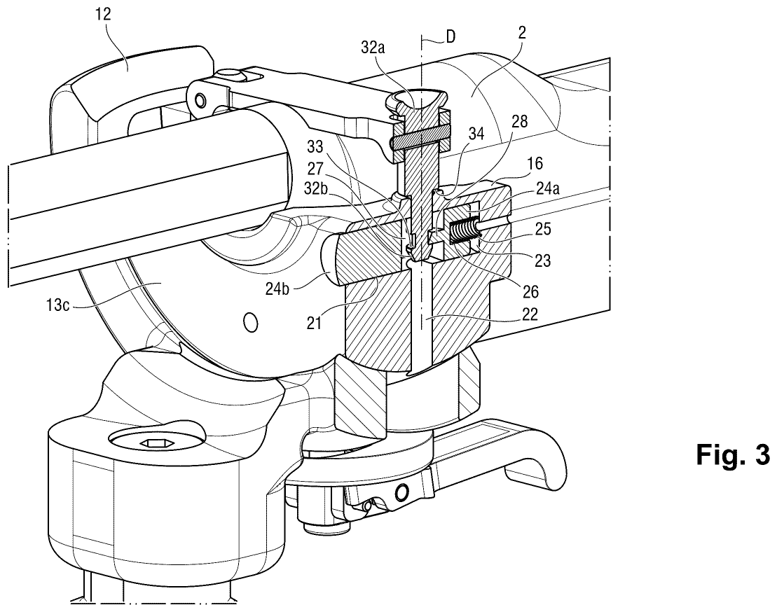

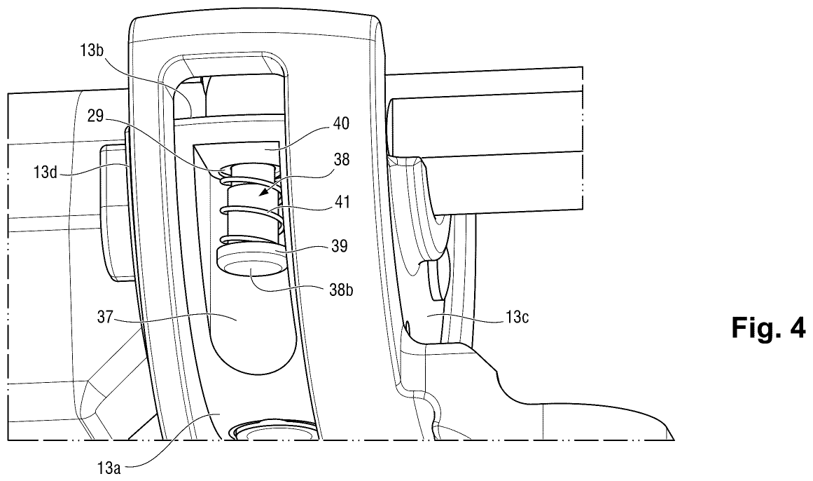

With reference to FIGS. 1 to 4, according to a first embodiment of the invention, the installation (denoted in general by reference numeral 1) comprises an examination apparatus 2 extending generally longitudinally along an axis Z in order to facilitate its insertion into the rectum of the patient.

Preferably, the apparatus 2 comprises a support 3, which extends in parallel with the axis Z, is intended to be gripped at its proximal end by an operator and is extended at its distal end by a niche 4 of the apparatus 2. Said niche 4 is itself extended by a medical imaging probe 5 of the apparatus 2, said probe 5 also extending along the axis Z. As a result, the apparatus 2 can be handled more easily by the operator because of the niche 4.

Here, said probe 5 is configured to allow three-dimensional images of the prostate and its environment to be taken. The probe 5 is a three-dimensional ultrasound probe. It goes without saying that this application is not limiting and the installation 1 could be used in combination with different types of probes such as two-dimensional ultrasound probes or magnetic resonance probes (e.g. MRI).

To facilitate the work of the operator, the installation 1 may comprise a co-manipulated system for the apparatus (not shown here). By way of example, the co-manipulated system may be a robot, an articulated arm, a fixed arm, etc.

The installation 1 also comprises a device 10 for holding the apparatus 2 on the co-manipulated system.

The holding device 10 thus comprises a base 11 for fastening to the co-manipulated system. The base 11 is attached, for example, by attachment means of the screw/nut type.

The holding device 10 further comprises a cradle 12 that is attached to the base 11 and preferably formed in one piece with the base 11.

The holding device 10 also comprises a shell 13 mounted on the cradle 12 so as to be pivotable about at least one axis A. Preferably, the holding device 10 is shaped such that the axes A and Z are parallel and more preferably coincide. Preferably, the shell 13 is movably mounted on the cradle 12.

The shell 13 and/or the cradle 12 comprise means (not referenced here) for guiding at least the pivoting of the shell 13 relative to the cradle 12 about the axis A. The shell 13 and/or the cradle 12 comprise stops (not referenced here) that limit the rotation of the shell 13 relative to the cradle 12.

Preferably, the shell 13 has at least one surface in contact with at least one surface of the cradle 12, said surfaces having complementary shapes. For example, the cradle 12 is shaped as a double arch, and the shell 13 received in the double arch is generally shaped as a cylinder segment (of a diameter similar to that of the double arch), for example as a half-cylinder.

More precisely, in this case the shell 13 has a curved lower face 13a (the face in contact with the double arch of the cradle 12), a planar upper face 13b and two side flanks 13c, 13d, each connecting the curved lower face 13a on one side and the planar upper face 13b on the other. The planar upper face 13b preferably has a recess 14 for receiving the apparatus 2, the receiving recess 14 therefore hollowing out the planar upper face 13b. Preferably, the receiving recess 14 is centred on the planar upper face 13b so that the axes A and Z coincide. On either side of the receiving recess 14, the planar upper face 13b therefore has two planar regions 15 and 16.

Furthermore, the holding device 10 comprises a first locking member 20 for temporarily securing the apparatus 2 to the shell 13.

For this purpose, the shell 13 has a first aperture 21 extending along an axis B that is parallel to the axis A and/or the axis Z. In this example, the first aperture 21 opens onto the first side flank 13c. However, the first aperture 21 does not open onto the second side flank 13d and therefore has a bottom 23. The shell 13 is also arranged such that the first side flank 13c is that facing the base 11.

In addition, the shell 13 comprises a second aperture 22 extending along an axis C, which in this case is orthogonal to the axis B. The second aperture 22 opens onto a first of the two planar regions 16.

The first locking member 20 comprises a latch 24 arranged in the shell 13, more precisely in the first aperture 21. The latch 24 therefore extends longitudinally along the axis B.

Preferably, the latch 24 is movably mounted in the first aperture 21, preferably is mounted in the first aperture 21 so as to be slidable along the axis B.

Also preferably, the first locking member 20 comprises a spring 25 which is connected at a first end to the bottom 23 and at a second end to the latch 24. For example, the latch 24 comprises a first opening 26 formed in its first longitudinal end 24a (i.e. the end facing the bottom 23 of the first aperture 21). The first opening 26 extends, for example, coaxially with the axis B. The first opening 26 opens only at the first longitudinal end 24a.

Furthermore, the latch 24 comprises a second opening 27 formed transversely in the latch 24. Here, the second opening 27 extends radially along an axis D. The second opening 27 also passes right through the latch 24. The second opening 27 does not, however, open into the first opening 26. The latch 24 is configured such that the axis B and the axis D are orthogonal to each other in this case and/or such that the axis D is parallel to the axis C.

The latch 24 comprises at least one catch 28 extending into the second opening 27. The catch 28 extends longitudinally in a manner tilted with respect to or parallel to the axis B. The catch 28 extends in a manner tilted with respect to or orthogonal to the axis D.

The latch 24 is configured such that when the spring 25 is in the position of equilibrium, its second longitudinal end 24b is outside the shell 13. The latch 24 is also configured such that when the spring 25 is in the position of equilibrium, the second aperture 22 and the second opening 27 are at least partially aligned such that the catch 28 extends at least partly into the second aperture 22.

In this position, an operator can press the second longitudinal end 24b from outside the shell 13 to move the latch 24 until the first longitudinal end 24a abuts the bottom 23. The spring 25 is then loaded, and the catch 28 is offset from the second aperture 22.

When the operator releases the latch 24, the spring 25 returns to its position of equilibrium, bringing the second longitudinal end 24b back to its initial position and the catch 28 back into the second aperture 22.

The first locking member 20 also comprises a lock 30, the lock 30 comprising an element 31 for interacting with the latch 24.

The interacting element 31 is shaped to be mounted in the first locking member 20 in a manner movable between an active position, in which said element is locked by the latch 24, and an inactive position, in which said element can be moved relative to the latch 24.

The interacting element 31 is mounted in the second aperture 22 so as to be movable between its two positions. For example, the interacting element 31 is mounted so as to be movable between its two positions by at least one translational movement along the axis C.

In this case, the interacting element 31 extends longitudinally along an axis that coincides with the axis C when the interacting element 31 is in its active position.

In this example, the interacting element 31 is in the form of a peg 32 comprising a first longitudinal end 32a and a second longitudinal end 32b.

The upper face of the first longitudinal end 32a is hollowed out and/or textured.

This makes it easier for an operator to handle the peg 32.

A groove 33 is formed in the lower part of the peg 32, the groove 33 being able to interact with the catch 28 to temporarily lock the peg 32 in the second aperture 22 (thus defining the active position of the interacting element 31), as will be described below.

Preferably, the second longitudinal end 32b has at least one slope that tapers axially away from the first longitudinal end 32a. This simplifies the insertion of the catch 28 into the groove 33 when the peg 32 is lowered into the second aperture 22.

For example, the second longitudinal end 32b is frustoconical. In this case, the groove 33 is formed above the truncated cone.

Preferably, the first locking member 20 and/or the shell 13 comprises a stop to limit the extent to which the peg 32 enters the second aperture 22. For example, the peg 32 has a shoulder 34 bearing against a flange 35 of the shell 13 that locally raises the first planar region 16 (the flange being arranged coaxially with the second aperture 22).

The peg 32, however, can in this case leave the second aperture 22 completely when it is in its inactive position.

The lock 30 also comprises an attachment 36 of the lock 30 to the shell 13.

The shell 13 here comprises a third aperture 29 extending along an axis E. The second aperture 29 opens into a cavity 37 formed on one of the sides of the shell 13. The third aperture 29 also opens onto the second planar region 15. As a result, the second aperture 22 and the third aperture 29 extend on either side of the receiving recess 14. In the present case, the axis E is parallel to the axis C.

The attachment 36 comprises, for example, a shaft 38 extending into the third aperture 29.

The shaft 38 is preferably shaped to be mounted in the third aperture 29 so as to be movable between a high position and a low position. For example, the shaft 38 is mounted so as to be movable between its two positions by a translational movement, preferably a translational movement along the axis E.

The shaft 38 preferably extends longitudinally along the axis E.

The second longitudinal end 38b of the shaft comprises a flange 39 which abuts a wall 40 of the cavity 37 to limit the travel of the shaft 38 in the third aperture 29. The wall/flange interaction defines the high position of the shaft 38.

Preferably, the attachment 36 also comprises a spring 41 attached to a first end of the shaft 38 and to a second end of the shell 13. Here, the spring 41 thus forms an elastically deformable region of the first locking member 20.

More precisely, the spring 41 is attached firstly to the second longitudinal end 38b of the shaft 38 and secondly to the wall 40 of the cavity 37.

The spring 41 is shaped so as to move the second longitudinal end 38b of the shaft 38 away from the wall 40 of the cavity 37. The position of equilibrium of the spring 41 thus defines the low position of the shaft 38.

The lock 30 further comprises an arm 42, which here is hinged at a first end to the attachment 36 and at a second end to the interacting element 31. More precisely, the arm 42 here is hinged at its first end to the shaft 38 (in particular to the first longitudinal end 38a of the shaft 38) and at its second end to the peg 32 (in particular to the first longitudinal end 32a of the peg 32). For example, each of the ends of the arm 42 is shaped as a fork joint, the arms of which are mounted on a hinge pin that passes through either the shaft 38 or the peg 32.

In particular, the arm 42 is hinged about an axis F on the attachment 36 and about an axis G on the interacting element 31. The axes F and G are preferably parallel to each other. The axis F is preferably parallel to the axis A and/or preferably orthogonal to the axis E. The axis G is preferably parallel to the axis A and/or preferably orthogonal to the axis C.

Preferably, the shell 13 is provided with a flange 43 that locally raises the second planar region 15, the flange 43 extending coaxially with the third aperture 29. A channel 44 is formed in the upper face of the flange 43, the channel 44 having a shape corresponding to that of the fork joint forming the first end of the arm 42.

As a result, the channel 44 makes it possible to guide the rotational movement of the arm 42 relative to the shell 13 about the axis F at least when the shaft 38 is in its low position.

It is further noted that the interaction between the channel 44 and the fork joint also makes it possible to define a stop for the shaft 38, thus preventing the shaft 38 from entering the shell 13. This stop also defines the low position of the shaft 38.

The arm 42 is also shaped so as to wedge the apparatus 2 against the shell 13.

Preferably, the arm 42 comprises a planar portion 45 bearing against a flat surface 46 of the apparatus 2.

This promotes the proper holding of the apparatus 2 owing to the flat-on-flat contact between the arm 42 and the apparatus 2.

During operation, the operator lifts the lock 30 and arranges the apparatus 2 such that it extends into the receiving recess 14.

The operator then lowers the lock 30 until the planar portion 45 bears against the flat surface 46 the apparatus 2. This illustrates the benefit of the attachment 36 sliding in the shell 13 owing to the spring 41, namely that the lock 30 can adjust to the various possible diameters of apparatuses 2 available on the market.

Next, the operator presses the latch 24 inside the first aperture 21 until the first longitudinal end 24a abuts the bottom 23, before pressing on the peg 32 to push it into the second aperture 22 until the shoulder 34 is in contact with the flange 35 (since the latch 24 has been pressed, the catch 28 extends out of the second aperture 22 and does not interfere with the movement of the peg 32 into the second aperture 22). The peg 32 is still in its inactive position.

The operator then releases the latch 24 while maintaining the pressure applied to the peg 32; the latch 24 returns to its original position (corresponding to the position of equilibrium of the spring 25), causing the catch 28 to be inserted into the groove 33. The peg 32 is then in its active position. The operator can therefore release the peg 32.

The apparatus 2 is thus held in the holding device 10 by being pressed between the arm 42 and the receiving recess 14, the lock 30 being locked in the shell 13 because the peg 32 is in the active position.

It is therefore clear that attaching the apparatus 2 to the shell 13 is simple and rapid.

Advantageously, the spring 41 tends to return the shaft 38 to its low position and therefore to press the arm 42 towards the receiving recess 14; this reduces the risk of the apparatus 2 accidentally coming out of the holding device 10.

To separate the apparatus 2 from the shell 13, the operator pushes the latch 24 back into the first aperture 21. The catch 28 is then removed from the groove 33. Under the action of the spring 41, the peg 32 rises into the second aperture 22, which moves it away from the second opening 27.

The peg 32 is thus in its inactive position, and the arm 42 can then be raised in order to separate the apparatus 2 from the shell 13. The operator can thus release the peg 32.

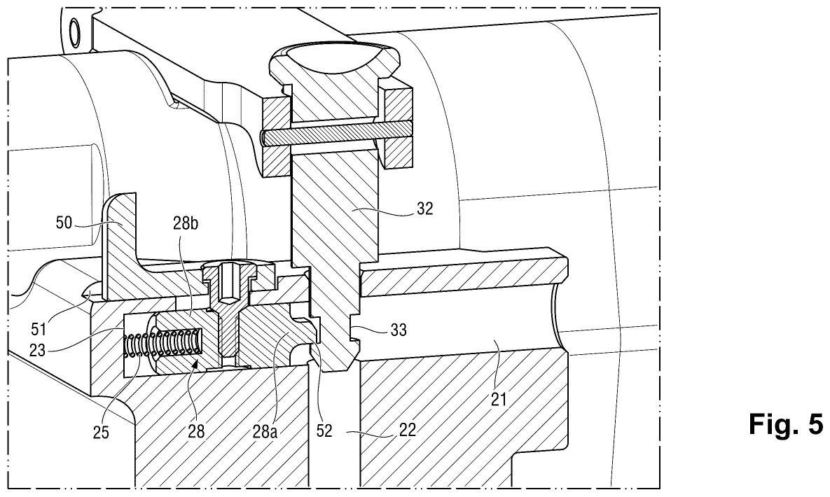

With reference to FIG. 5, the second embodiment is identical to the first embodiment except in the region of the latch 24.

In the second embodiment, the latch 24 thus comprises a handle 50 for handling a catch 28.

The handle 50 is mounted so as to be movable relative to the shell 13 between a retracted position and an extended position. By way of example, the handle 50 is mounted so that it can move in translation between its two positions. For example, a channel 51 is formed in the first planar region 16 to allow the handle 50 to slide along this channel 51. Optionally, the channel extends in parallel with the axis Z and/or the axis A.

The handle 50 is attached to the catch 28, for example by a screw or any other attachment means. Alternatively, the handle 50 and the catch 28 are formed in one piece.

The catch 28 is thus also mounted so that it can move in translation in parallel with the axis Z and/or the axis A relative to the shell 13. For example, the catch 28 slides inside the first aperture 21.

The catch 28 has a first longitudinal end 28a provided with at least one stud 52 intended to be inserted into the groove 33 in the peg 32 (as in the first embodiment).

The spring 25 of the latch 24 is attached at one end to the second longitudinal end 28b of the catch 28 and at its other end to a bottom 23 of the first aperture 21. In the second embodiment, the spring 25 is arranged on the same side of the shell 13 as the part of the latch 24 handled by the operator (here the handle 50), whereas in the first embodiment the spring 25 was arranged on the opposite side of the shell 13, where the part of the latch 24 handled by the operator was located (here the second longitudinal end 24b of the latch 24).

When the operator wishes to place the peg 32 in its active position, they pull the handle 50 to force the catch 28 to move back towards the bottom 23. The stud 52 is thus offset from the second aperture 22. The operator can then push the peg 32 into the second aperture 22 until the shoulder 34 is in contact with the flange 35.

The operator can then release the pressure applied to the handle 50 while still pressing the peg 32; under the action of the spring 25, the catch 28 returns to the original position and thus enters the groove 33, locking the peg 32 in the second aperture 22. The peg 32 is then in its active position.

When the operator wishes to return the peg 32 to its inactive position, they pull the handle 50 again to force the catch 28 to disengage from the groove 33. Under the action of the spring 41, the peg 32 rises in the second aperture 22 out of the reach of the catch 28. The peg 22 is then in its inactive position.

The operator then releases the pressure applied to the handle 50; under the action of the spring 25, the catch 28 returns to its original position.

Apart from what has been stated above, everything set out for the first embodiment is also applicable here.

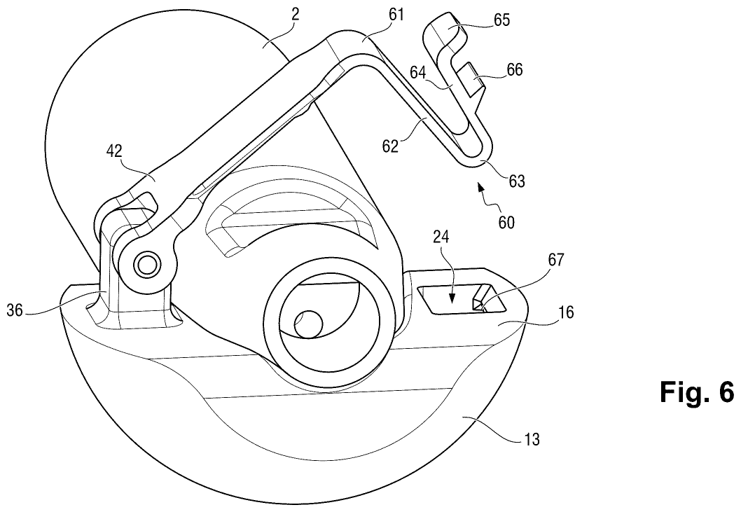

With reference to FIG. 6, the second embodiment is identical to the first embodiment except in the region of the latch 24, the interacting element 31 and the attachment 36.

In this case, the attachment 36 is a simple connector rigidly connected to the shell 13 (and optionally formed in one piece with the shell 13) on which the arm 42 is hinged.

Here, the interacting element 31 is shaped as a tab 60 that snaps into a latch 24, which is shaped as a recess formed in the shell 13. The tab 60 is not hinged to the arm 42 as in the other embodiments but rather is formed in one piece with said arm 42.

In the present case, the tab 60 is shaped so as to have at least one first return section 61 extending the arm 42, followed by a first planar portion 62, followed by a second return section 63, followed by a second planar portion 64 followed by a third return section 65.

The first planar portion 62 is preferably orthogonal to or tilted relative to the arm 42.

The second planar portion 64 is preferably tilted relative to the first planar portion 62. The second planar portion 64 is thus tilted so as to extend in the opposite direction of the arm 42.

The tab 60 is thus generally V-shaped.

It goes without saying that the tab 60 is flexible to ensure the snap-in engagement in the latch. The tab 60 thus forms an elastically deformable region of the first locking member 20.

Preferably, the second planar portion 64 is provided with a lug 66. The lug 66 is, for example, shaped so as to extend transversely on the second planar portion 64, optionally over the entire width of the second planar portion 64.

The latch 24 opens into a cavity formed on one of the sides of the shell 13. The latch 24 also opens onto the second planar region 16.

When the operator wishes to place the interacting element 31 in its active position, they thus press on the tab 60, pushing it into the latch 24 until the free end of the interacting element 31 (here the third return section 66) rests on the second planar region 16. In this position, the lug 66 then bears against a wall 67 of the cavity, said wall 67 being arranged under the second planar face 16.

The tab 60 thus grips a body of the shell 13 delimited at the top by the second planar region 16 and at the bottom by the wall 67. This also moves the two planar portions 62, 64 of the tab 60 further apart.

This allows the holding of the interacting element 31 in its active position to be supported.

It is also noted that the first planar portion 62 is then pressed against a side flank 68 of the latch 24. This also moves the two planar portions 62, 64 of the tab 60 further apart.

This allows the holding of the interacting element 31 in its active position to be supported.

To return to the inactive position of the interacting element 31, the operator applies a force to bring the two planar portions 62, 64 of the tab 60 closer together in order to be able to remove it from the latch 24.

Apart from what has been stated above, everything set out for the first embodiment is also applicable here.

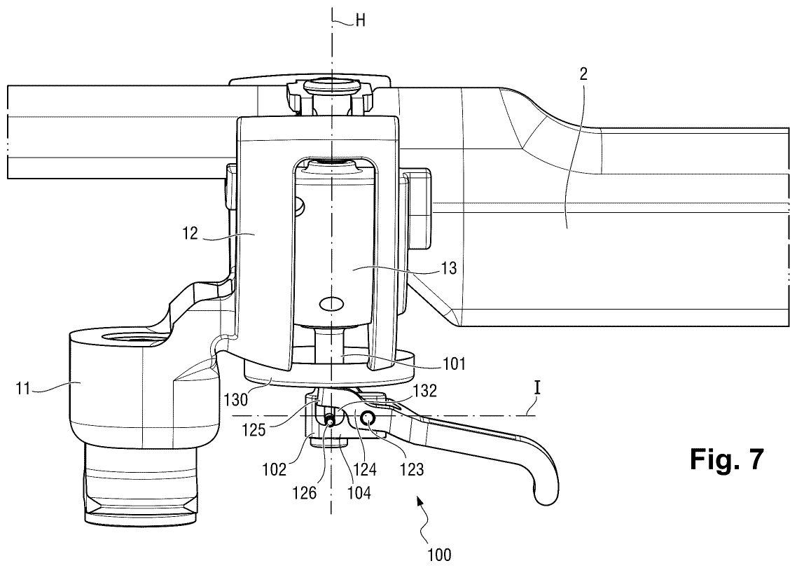

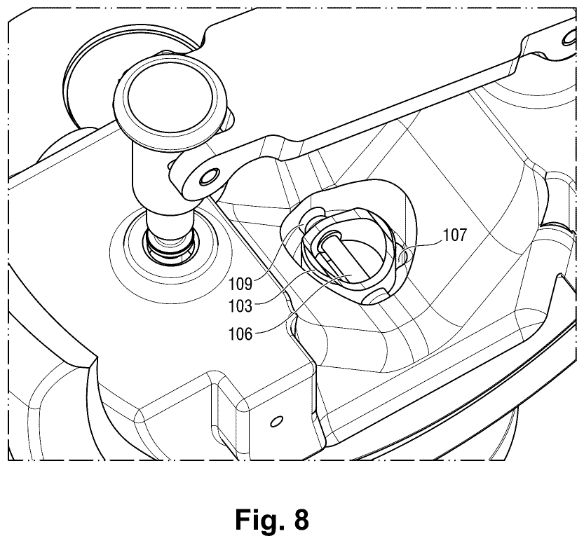

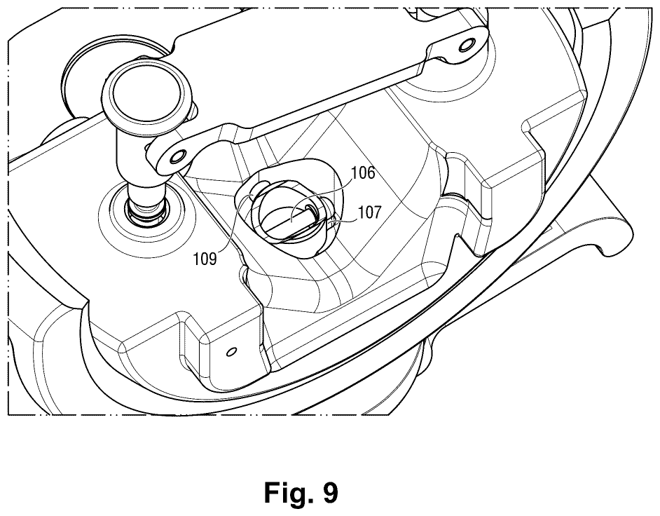

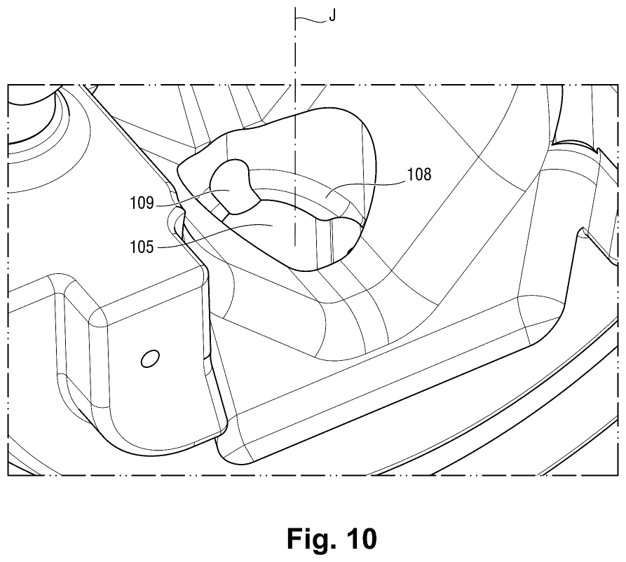

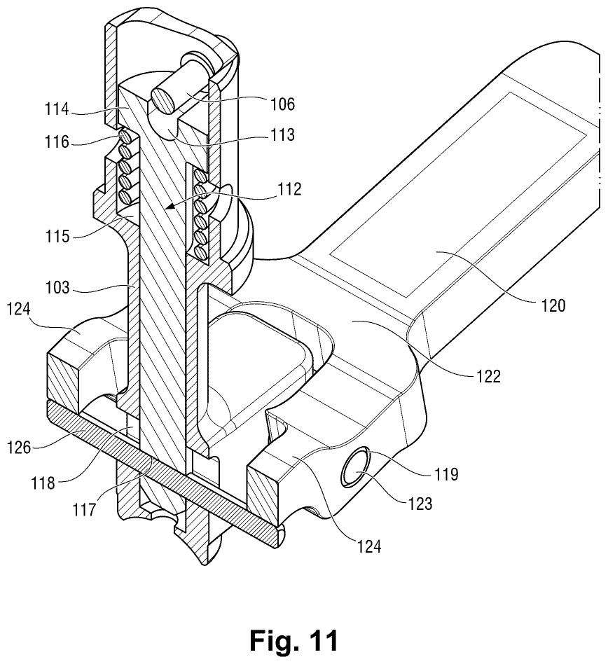

With reference to FIGS. 7 to 11, a second locking member 100 of the holding device 10 will now be described in connection with the first embodiment. It goes without saying that this application is not limiting and the second locking member 100 described below is also applicable to the second embodiment or to the third embodiment.

The second locking member 100 firstly comprises a portion 101 for connecting to the shell 13.

The connection portion 101 here comprises a hollow casing 102 having a column 103 extending longitudinally along an axis H and a base 104 that extends the column at the lower end. The base extends longitudinally along an axis I, which is orthogonal to the axis H in this case. The column 103 and the base 104 are preferably formed in one piece.

The shell 13 comprises a fourth aperture 105 which extends longitudinally along an axis J. The axis J is parallel to the axis E in this case. The fourth aperture 105 extends so as to open at both its ends out of the shell 13. More precisely, here the fourth aperture 105 extends so as to open onto the curved lower face 13a of the shell 13 at one end and into the receiving recess 14 at the other end. The fourth aperture 105 is preferably centred in the shell 13. Here, the fourth aperture 105 extends orthogonally to the axis A.

The column 103 is therefore arranged in the shell 13 so as to extend into the fourth aperture 105. The column is thus arranged such that the axes H and J then coincide (i.e. the column 103 and the fourth aperture 105 extend coaxially).

The column 103 is mounted in the shell 13 so as to be movable between a working position, in which it allows the second locking member 100 to be secured to the shell 13, and a rest position, in which it allows the second locking member 100 to be separable from the shell 13.

As a result, the second locking member 100 can be completely removed from the shell 13, making it easier to clean between two operations.

For example, the column 103 is mounted so that it can move in translation and/or in rotation in the fourth aperture 105, preferably so that it can move in rotation and/or in translation relative to the axis J.

More precisely, here the column 103 is first mounted so that it can move in translation along the axis J until it reaches its rest position. The column 103 is then mounted so that it can move in rotation and in translation (in a helical movement) relative to the axis J between its rest position and its working position.

Preferably, the column 103 and/or the shell 13 comprise means for guiding the column 103 in the shell 13.

Optionally, the guide means comprise a guide peg 106 borne by the column 103 and attached to said column 103. In this case, the peg 106 extends transversely in the column 103. Here, the peg 106 extends radially along an axis K, i.e. orthogonally to the axis H. In this case, the peg 106 extends at the top end of the column 103. The peg 106 is also shaped radially so as to extend outside the column 103 on either side thereof. The peg 106 thus projects at both its ends from the column 103.

In a corresponding manner, the guide means comprise channels 107 that extend in the fourth aperture 105 along the axis J, each channel 107 slidably receiving one of the ends of the peg 106. Each channel 107 extends in the fourth aperture 105 from the curved lower face 13a to a helix pitch 108 formed by a first internal shoulder of the fourth aperture 105 (the first shoulder forming part of the guide means). The top end of each channel 107 thus defines the rest position of the column 103. The helix pitch 108 extends, for example, over a quarter turn of the fourth aperture 105.

Each helix pitch 108 thus extends between one of the upper ends of the channels 107 and terminates in a recess 109. Each recess 109 is thus shaped to receive one of the ends of the peg 106 when the column 103 is in the working position. The recesses 109 thus define the working position of the column 103.

Preferably, the guide means also comprise an additional stop between the column 103 and the shell 13, defining the working position of the column 103. For example, the column 103 comprises an external collar that abuts a second shoulder formed in the fourth aperture 105.

During operation, the column 103 first slides along the fourth aperture 105 (owing to interaction between the peg 106 and the channels 107) and then performs a helical movement (owing to interaction between the peg 106 and the screw pitches 108) until the peg 106 fits in the recesses 109. In this position, the column 103 can no longer descend along the fourth aperture 105, thus temporarily securing the shell 13 to the column 103.

To separate the column 103 from the shell 13, it is enough to apply a rotational force to the column 103 in order to bring the peg 106 out of the recesses 109 and thus lower the peg 106 as far as the channels 107. The column 103 can then be pulled entirely towards the outside of the shell 13 by translation.

Preferably, the column 103 and/or the fourth aperture 105 are shaped so that at least part of the upper end of the column 103 bears against the apparatus 2 when the column 103 is in the working position.

This makes it possible to reduce the risk of the peg 106 accidentally coming out of the recesses 109 while the apparatus 2 is in use.

Preferably, the column 103 and/or the fourth aperture 105 are shaped to match the shape of the region of the apparatus 2 that bears against the column 103 when it is in its high position. For example, the upper end of the fourth aperture 105 is frustoconical and/or the upper end of the column 103 is frustoconical while the apparatus 2 has a V-shaped lower face in contact with the fourth aperture 105 and/or the column 103.

The connection portion 101 also comprises a shaft 112 arranged in the casing 102, more particularly in the column 103. The shaft 112 extends longitudinally in a direction that preferably coincides with the axis H. The shaft 112 and the column 103 thus extend coaxially.

The shaft 112 is mounted in the column 103 so as to be movable between a low position and a high position. The shaft 112 is, for example, mounted so that it can move in translation between its low position and its high position, preferably so that it can move in translation along the axis H.

The shaft 112 and the column 103 are provided with means for temporarily securing them together when the shaft 112 is in its high position.

For example, the means for temporarily securing them together comprise male and female members, one of which is borne by the shaft 112 and the other by the column 103, respectively, and one of which optionally is borne by the top end of the shaft 112 and the other by the top end of the column 103.

By way of example, in this case the male member is formed directly by the aforementioned peg 106.

By way of example, the female member is a channel 113 that is formed in a top end of a head 114 of the shaft 112 (forming the top end of the shaft) and capable of receiving the peg 106, which in this case forms the male member. Here, therefore, the channel 113 extends transversely in the shaft 112. The channel 113 thus extends radially, i.e. orthogonally to the axis H.

The lower end of the head 114 is shaped to abut an internal shoulder 115 of the column 103. This head/shoulder interaction makes it possible to define the low position of the shaft 112.

Preferably, the connection portion 101 also comprises a first spring 116, a first end of which is connected to the shaft 112 and a second end of which is thus connected to the column 103. More specifically, here the first end of the first spring 116 is connected to the head 114 (e.g. to the lower end of the head 114), and the second end of the first spring 116 is connected to the internal shoulder 115. The first spring 116 is thus configured to push the shaft 112 towards its high position.

An aperture 117 is formed in the lower end of the shaft 112. The aperture 117 extends transversely into the shaft 112 so as to open at both its ends outside the shaft 112. The aperture 117 extends radially into the shaft 112, i.e. orthogonally to the axis H.

In a corresponding manner, an aperture 118 arranged facing the aperture 117 is formed in the base 102. The aperture 118 extends transversely into the base 102 so as to open at both its ends outside the base 102. The aperture 118 extends radially into the base 102, i.e. orthogonally to the axis H. The aperture 118 is configured to allow the shaft 112 to slide in the column 103 between its high position and its low position. For this purpose, the aperture 118 has an oblong cross section, the oblong cross section extending vertically in this case. The upper and lower ends of the oblong cross section thus define the high and low positions of the shaft 112.

Furthermore, a second aperture 119 is formed in the base 102. The aperture 119 extends transversely into the base 102 so as to open at both its ends outside the base 102. The aperture 119 extends radially into the base 102, i.e. orthogonally to the axis H. The two apertures 118, 119 in the base 102 thus extend in parallel with each other.

The second locking member 100 also comprises a handling portion.

The handling portion here comprises a handle 120, which has a proximal end that can be handled by the operator, and which is extended at its distal end by a fork joint 122. The fork joint 122 and the handle 120 are preferably formed in one piece.

The fork joint 122 is hinged to the base 102 by means of a hinge pin 123 passing through the aperture 119. In a manner known per se, the fork joint 122 has two branches 124, each branch 124 being pivotably mounted on the hinge pin 123 on either side of the base 102.

In particular, each branch 124 is extended by a finger 125 that forms the free end of the branch 124 (the finger 125 being formed in one piece with the branch 124).

Here, each finger 125 interacts with the same hinge pin 126 arranged in the aperture 118 of the base 102 and the aperture 117 facing the shaft 112. In this case, the two hinge pins 123, 126 extend in parallel with one another.

The second locking member 100 further comprises a lock 130 arranged between the connection portion 101 on one side and the handling portion on the other.

The lock 100 firstly comprises an elastically deformable element, for example a spring washer 131. An aperture through which the column 103 (and thus the shaft 112) extends is formed in the washer 131. The aperture preferably passes through the washer 131 at its centre such that the washer 131 is coaxial with the shaft 112 and the column 103. The aperture is larger than the diameter of the column.

The washer 131 is intended to bear against the double arch outlined by the cradle 12.

Preferably, the washer 131 is a conical washer. The washer 131 is also arranged such that its upper face (i.e. the face intended to come into contact with the cradle 12) is the face having the widest cross section.

This makes it possible to increase the exchange surfaces between the washer 131 and the cradle 12 when the washer 131 bears against the cradle 12.

The lower face of the washer 131 is extended on either side of the lower face by two tabs 132, each secured to the hinge pin 126. An aperture having a cross section similar to that of the hinge pin 126 is thus formed in each of the tabs 132. In this case, the two tabs 132 extend in parallel with one another.

It is therefore understood that the tabs 132 externally surround the column 103 on either side, which itself surrounds the shaft 112.

The hinge pin 126 is of such a length that it protrudes not only beyond the shaft 112 and the column 103 but also beyond the tabs 132. It is noted that the hinge pin 126 is rigidly connected to the shaft 112.

The fingers 125 themselves externally surround the base 102 and also the two tabs 132 on either side of the base 102 and the tabs 132. The fingers 125 thus extend between the washer 131 and the hinge pin 126.

The second locking member 100 is shaped such that the fingers 125 can interact with the free ends of the hinge pin 126 to lower the hinge pin 126 so far as to abut the bottom of the aperture 128 when the handle 120 is raised. During operation, to secure the second locking member 100 to the shell 13, the operator inserts the connection portion 101 into the fourth aperture 105 (the shaft 112 is then in the high position such that it is rigidly connected to the casing 101 by the interaction between the channel 113 and peg 106). The operator then performs a helical movement using the handle 120, which brings the peg 106 into the recesses 19, thus fixing the second locking member 100 in place in the shell 13.

When the handle 120 is in the lowered position, which is the position of equilibrium of the second locking member 100, the shaft 112 is in its high position and the washer 131 is squeezed against the cradle 12, temporarily securing the shell 13 and the cradle 12.

To move the shell 13 relative to the cradle 13 about the axis Z as a far as a target relative position, the operator raises the handle 120; as the handle 120 pivots, the fingers 125 press against the hinge pin 126, which is lowered, thus causing the shaft 112 to move from its high position to its low position. This then causes a downward translation of the washer 131 (since it is connected to the hinge pin 126). The washer 131 is thus offset from the cradle 12 such that the shell 13 can be moved relative to the cradle 12 by means of the handle 120.

Once the target position is reached, the operator releases the handle 120; under the action of the first spring 116, the shaft 112 naturally returns to its high position. The corresponding sliding of the hinge pin 126 also causes the washer 131 to slide upwards, which is then squeezed against the cradle 12 again. The shell 13 and the cradle 12 are thus once again secured together.

Advantageously, the position of equilibrium of the second locking member 130 corresponds to the state in which the cradle 12 and the shell 13 are secured together, which is more secure.

It goes without saying that the invention is not limited to the described embodiments, and alternative embodiments may be provided without departing from the scope of the invention.

Thus, the interaction between the interacting element and the latch could be different from that described. For example, the latch could comprise the groove and the interacting element could comprise the catch.

The end of the latch on which the operator presses could also be hollow and/or textured.

The holding device could comprise an additional piece that holds the peg in position in the recesses of the second locking member throughout the time the apparatus is in use.

The locking member for securing the examination apparatus to the shell during operation could be a prior-art attachment system (for example a wing screw).

1. A device for holding a medical examination apparatus, the device comprising at least a cradle, a shell mounted on the cradle so as to be movable about an axis of rotation, and a locking member for temporarily securing the cradle to the shell, characterised in that the locking member comprises at least:
a portion for connecting to the shell,
a lock comprising at least one elastically deformable element,
a handling portion connected to the connection portion and to the lock so as to be able to move the elastically deformable element between an unlocked position, in which said element is offset from the cradle, and a locked position, in which the element presses against the cradle.

2. The device according to claim 1, wherein the elastically deformable element is a washer

3. The device according to claim 1, wherein the elastically deformable element is mounted on a hinge pin on which the handling portion acts in order to move the elastically deformable element between its two positions.

4. The device according to claim 1, wherein the handling portion is pivotably mounted on the connection portion

5. The device according to claim 1, wherein the handling portion comprises a handle extended by a fork joint secured to the handling portion.

6. The device according to claim 1, wherein the connection portion comprises a hollow casing and a shaft mounted in the hollow casing.

7. The device according to claim 6, wherein the shaft is mounted in the casing so as to be slidable between a first position and a second position.

8. The device according to claim 6, wherein the shaft is rigidly connected to a hinge pin on which the elastically deformable element is mounted.

9. The device according to claim 6, wherein the casing is provided with an aperture through which the hinge pin rigidly connected to the shaft extends.

10. The device according to claim 6, comprising a spring connected both to a column of the casing and to the shaft.

11. The device according to claim 6, wherein the shell is provided with an aperture through which the column and the shaft extend.

12. The device according to claim 11, wherein the aperture and the column are provided with their temporary securing means.

13. The device according to claim 12, wherein the temporary securing means comprise a peg and at least one recess in which the peg is able to fit temporarily.

14. The device according to claim 1, comprising a second locking member for securing the examination apparatus to the shell during operation, the second locking member comprising at least a latch and a lock, the lock comprising at least:
an attachment to the shell,
an element for interacting with the latch, the interacting element being mounted in the device so as to be movable between an active position, in which said element is locked by the latch, and an inactive position in which said element is not locked by the latch,
an arm which is connected at a first end to the attachment and connected at a second end to the interacting element, the arm being intended to hold the examination apparatus against the shell.

15. An installation comprising a the device according to claim 1.
